(19) United States
(12) Patent Application Publication
(10) Pub. No.: US 2015/0231045 A1
(43) Pub. Date: Aug. 20, 2015

(54) ANTI-DANDRUFF COMPOSITION COMPRISING 1-ACETOXYCHAVICOL ACETATE

(71) Applicants: AnalytiCon Discovery GmbH, Potsdam, (DE); BRAIN AG - Biotechnology Research and Information Network AG, Zwingenberg (DE)

(72) Inventors: Michael Krohn, Lorsch (DE); Jörg Mampel, Bensheim-Auerbach (DE); Lars Ole Haustedt, Potsdam (DE); Grit Kluge, Trebbin (DE)

(73) Assignees: AnalytiCon Discovery GmbH, Potsdam, (DE); BRAIN AG - Biotechnology Research and Information Network AG, Zwingenberg (DE)

(21) Appl. No.: 14/426,352

(22) PCT Filed: Sep. 6, 2013
(86) PCT No.: PCT/EP2013/068513

§ 371 Date: Mar. 5, 2015

Related U.S. Application Data
(60) Provisional application No. 61/697,883, filed on Sep. 7, 2012.

(30) Foreign Application Priority Data
Sep. 7, 2012 (EP) 12183589.6

(51) Int. Cl. A61K 8/37; A61K 8/97; A61Q 5/00

(52) U.S. Cl. CPC A61K 8/37; A61Q 5/006; A61K 8/97; A61K 2800/805; A61K 2800/52

(57) ABSTRACT

The present invention relates to an anti-dandruff composition comprising 1-acetoxychavicol acetate (I). Furthermore, the present invention relates to a method for preparing said anti-dandruff composition and the use of said anti-dandruff composition for treating or preventing Malassezia induced dandruff formation. Moreover, the present invention relates to a method for preparing an Alpinia galanga extract and an extract obtainable or obtained by said method as well as the use of this extract for treating Malassezia induced dandruff formation comprising applying an anti-dandruff composition comprising said extract.

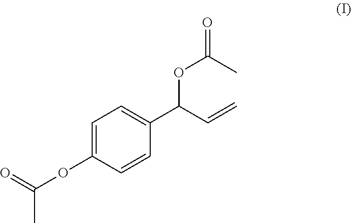 [SMILES: C=CC(OC(C)=O)c1ccc(OC(C)=O)cc1]

The present invention relates to an anti-dandruff composition comprising 1-acetoxychavicol acetate (I). Furthermore, the present invention relates to a method for preparing said anti-dandruff composition and the use of said anti-dandruff composition for treating or preventing Malassezia induced dandruff formation. Moreover, the present invention relates to a method for preparing an Alpinia galanga extract and an extract obtainable or obtained by said method as well as the use of this extract for treating Malassezia induced dandruff formation comprising applying an anti-dandruff composition comprising said extract.

Dandruff is one of the most frequent skin problems, affecting more than 50% of men and women worldwide. The formation of dandruff can be triggered by different factors such as increased skin oil production commonly referred to as sebum or sebaceous secretions, by functional disturbances of the sebaceous glands caused by hereditary factors, by diseases of internal organs or a disturbance in their functioning, as a side-effect of medicaments, by stress, strain on the nerves and psychological problems and by drying out of the scalp by unsuitable care products, bacteria and fungi (see US 2010/0215775 A1).

Dandruff in most cases forms as a result of overproduction of horny cells. This overproduction is triggered by tiny centers of inflammation of the scalp, which are visible only under a microscope. Because of the inflammation, the horny cells of the scalp do not mature fully, so that they are shed prematurely in large cell structures—as dandruff.

A frequent cause is the increased colonization with bacteria or fungi, such as with the fungus Malassezia furfur (previously known as Pityrosporum ovale) or Malassezia globosa (see Gupta A K et al. (2004). “Skin diseases associated with Malassezia species”. J. Am. Acad. Dermatol. 51 (5): 785-98), that metabolizes triglycerides present in sebum by the expression of lipase, resulting in a lipid byproduct oleic acid (OA).

During dandruff, the levels of Malassezia increase by 1.5 to 2 times of its normal level (see Ranganathan S, Mukhopadhyay T. (2010), Indian J Dermatol 55 (2): 130-134) Penetration by oleic acid through the top layer of the epidermis, the stratum corneum, results in an inflammatory response in susceptible persons which disturbs homeostasis and results in erratic cleavage of stratum corneum cells.

This increased growth of Malassezia species, such as, for example, M. globosa and M. furfur, can thus be regarded as one of the main causes of dandruff formation accompanied by considerable redness and itching of the scalp. Redness of the scalp and itching can be encouraged still further by increased sebum production (seborrhoea). In particular, the itching or tingling of the skin accompanying dandruff formation is generally considered disturbing. Since the itching frequently triggers a scratching reaction, it can lead to a further injury of the concerned skin area. Because of that as an additional complication infections with pathogenic excitation can also occur.

Inhibiting the growth of fungi of the genus Malassezia is one of the main methods of controlling dandruff formation. An important group of active ingredients that are currently being used in the treatment of dandruff are antimycotics for topical and systemic administration from the group of the azoles. Anti-dandruff agents known in the art are climbazole as well as further azoles such as, for example, benzimidazole, benzothiazole, bifonazole, butaconazole nitrate, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, thioconazole, as well as diazoles and triazoles, such as, for example, terconazole and itraconazole, as well as any desired combinations of the mentioned azoles.

However, various antimycotics known in the art are disadvantageous in that e.g. sensitization reactions are observed more frequently in clinical practice, but also because of the formation of potentially resistant strains.

Furthermore use of several anti-dandruff agents known in the art is accompanied with considerable skin irritation, e.g. owing to their pronounced reduction of the pH value of the skin. Thus, they are not capable of reducing the itching of the skin but instead may lead to a further injury of the skin area ether due to the triggered scratching reaction and/or as a result of the skin irritation. Further for anti-dandruff agents, such as coal tar, cancerogenic side-effects have been postulated.

There is thus still the need for advantageous anti-dandruff agents which are active against Malassezia species and at the same time mild and tolerated by the skin, in particular which are non-irritant, non-caustic, non-sensitizing and/or non-cancerogenic and preferably act at a skin-friendly pH value and are capable of alleviating the itching of the treated skin area.

### SUMMARY OF THE INVENTION

The present invention relates to an anti-dandruff composition comprising 1-acetoxychavicol acetate according to formula (i), in particular for use in treating or preventing Malassezia induced dandruff formation and/or Malassezia induced itching of the skin itching of the skin.

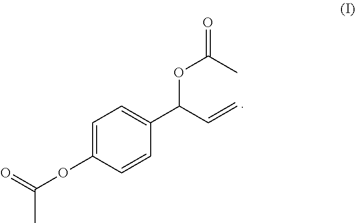 [SMILES: C=CC(OC(C)=O)c1ccc(OC(C)=O)cc1]

In a further aspect, the present invention relates to a method for the preparation of an Alpinia galanga extract and an Alpinia galanga extract obtainable or obtained by said method, comprising
(a) providing Alpinia galanga, more preferably a root of Alpinia galanga, and contacting the Alpinia galanga with a liquid S1 thereby forming a liquid phase L1 and a solid residue R0;
(b) separating L1 from R0;
(c) optionally drying L1 to give a residue R1;
(d) optionally dissolving the R1 in a liquid S2
   to give the Alpinia galanga extract.

In a further aspect, the present invention relates to a method for the preparation of an Alpinia galanga extract comprising 1-acetoxychavicol acetate, and an Alpinia galanga extract obtainable or obtained by said method, the method comprising
(a1) providing Alpinia galanga, more preferably the a root of Alpinia galanga, and contacting the Alpinia galanga with oil seeds;
(a2) subjecting the mixture according to (a1) to co-pressing to give a liquid phase L1 and a solid residue R0,
(a3) separating L1 from R0
   to give the Alpinia galanga extract.

The present invention furthermore relates to an anti-dandruff composition comprising 1-acetoxychavicol acetate according to formula (I) for use in treating or preventing Malassezia furfur induced dandruff formation and/or Malassezia furfur induced itching of the skin. In a further aspect the present invention relate to the use of an anti-dandruff composition comprising 1-acetoxychavicol acetate according to formula (I) for treating or preventing Malassezia induced dandruff formation. Furthermore, the present invention relates to a method of treating Malassezia induced dandruff formation comprising applying an anti-dandruff composition comprising 1-acetoxychavicol acetate according to formula (I).

It was surprisingly found that 1-acetoxychavicol acetate (I) is highly active against Malassezia species and at the same time mild and tolerated by the skin. Further, it was surprisingly found that, in contrast to other anti-dandruff agents described in the art, 1-acetoxychavicol acetate (I) clearly alleviates the itching of the skin. Further, the 1-acetoxychavicol acetate (I) is advantageous in that it can be easily obtained from a biosource, that is Alpinia galanga. Thus, it is contemplated that this substance is non-toxic, in particular when topically administered.

The term “ Alpinia galanga extract ” as used herein means a substance or composition obtained from Alpinia galanga extract by extraction, maceration or percolation of Alpinia galanga material with a suitable solvent and, optionally, by partial or complete removal of the solvent or by co-pressing the Alpinia galanga with suitable oil seeds. Thus, extracts in accordance with this invention are either so-called co-pressed extracts or solvent-processed fluid extracts or so called dry Alpinia galanga extracts obtained by evaporation of the whole liquid extract to dryness, e.g. by air drying, spray drying, vacuum oven drying, fluid-bed drying or freeze-drying, and optional washing and/or re-dissolving of this dry extract in at least one suitable solvent. Solvents suitable for extraction, percolation or maceration are known to those experienced in the art. Alkanes, alkanols, water, acetone and mixtures thereof as well as plant oils are particularly suited. Carbon dioxide in fluid or super-critical form and pressurized gases with solvent properties are also suitable as extraction agents.

According to one embodiment of the present invention, the acetoxychavicol acetate according to formula (I) is obtained from an Alpinia galanga extract which is obtained or obtainable by any one of the methods described above. According to a further, more preferred embodiment, the composition comprises an Alpinia galanga extract, as described above, which in turn comprises the acetoxychavicol acetate according to formula (I).

Thus, in one aspect, the present invention also describes an anti-dandruff composition as described above comprising 1-acetoxychavicol acetate according to formula (I) obtained from an Alpinia galanga extract. In this context of the present invention, the term “the 1-acetoxychavicol acetate according to formula (I) obtained from an Alpinia galanga extract” is denoted to mean that the 1-acetoxychavicol acetate (I) is isolated from an Alpinia galanga extract.

Thus, the present invention also describes a method for the preparation of an anti-dandruff composition comprising acetoxychavicol acetate according to formula (I), and an anti-dandruff composition obtained or obtainable by said method, in particular for use in treating or preventing Malassezia induced dandruff formation and/or Malassezia induced itching of the skin, said method comprising
(a) providing Alpinia galanga, more preferably a root of Alpinia galanga, and contacting the Alpinia galanga with a liquid S1 thereby forming a liquid phase L1 comprising 1-acetoxychavicol acetate (I) and a solid residue R0;
(b) separating L1 from R0;
(e) isolating acetoxychavicol acetate (I) from L1
(f) adding at least one suitable cosmetically and/or pharmaceutically acceptable excipient or carrier.

Further, the present invention also describes a method for the preparation of an anti-dandruff composition comprising 1-acetoxychavicol acetate according to formula (I), and an anti-dandruff composition obtained or obtainable by said method, in particular for use in treating or preventing Malassezia induced dandruff formation and/or Malassezia induced itching of the skin, said method comprising
(a1) providing Alpinia galanga, more preferably the a root of Alpinia galanga, and contacting the Alpinia galanga with oil seeds;
(a2) subjecting the mixture according to (a1) to co-pressing to give a liquid phase L1 comprising 1-acetoxychavicol acetate (I) and a solid residue R0,
(a3) separating L1 from R0
(e) isolating 1-acetoxychavicol acetate (I) from L1
(f) adding at least one suitable cosmetically and/or pharmaceutically acceptable excipient or carrier.

The isolation may be carried out by any suitable method known to those skilled in the art, such as crystallization, chromatography and the like.

According to preferred embodiment, the composition comprises an Alpinia galanga extract, as described above, which in turn comprises the acetoxychavicol acetate according to formula (I).

Thus, the present invention also relates to an anti-dandruff composition, in particular for use in treating or preventing Malassezia induced dandruff formation and/or Malassezia induced itching of the skin, the anti-dandruff composition comprising an Alpinia galanga extract which comprises said 1-acetoxychavicol acetate (I). Further, the present invention relates to a method for preparing the same, said method, comprising
(a) providing Alpinia galanga, more preferably a root of Alpinia galanga, and contacting the Alpinia galanga with a liquid S1 thereby forming a liquid phase L1 comprising the 1-acetoxychavicol acetate (I), and a solid residue R0;
(b) separating L1 from R0;
(c) optionally drying L1 to give a residue R1;
(d) optionally dissolving the R1 in a liquid S2
(f) adding at least one suitable cosmetically and/or pharmaceutically acceptable excipient or carrier.

In a further aspect, the present invention relates to a method for the preparation of a composition comprising an Alpinia galanga which comprises said 1-acetoxychavicol acetate (I), said method, comprising
(a1) providing Alpinia galanga, more preferably the a root of Alpinia galanga, and contacting the Alpinia galanga with oil seeds;
(a2) subjecting the mixture according to (a1) to co-pressing to give a liquid phase L1 comprising the 1-acetoxychavicol acetate (I), and a solid residue R0,
(a3) separating L1 from R0
   to give the Alpinia galanga extract.
(f) adding at least one suitable cosmetically and/or pharmaceutically acceptable excipient or carrier

The Alpinia galanga extract comprises 1-acetoxychavicol acetate (I) preferably in an amount the range of from 0.1 to 10% by weight, more preferably in an amount in the range of from 0.1 to 5% by weight, more preferably in an amount in the range of from 0.3 to 3% by weight, more preferably in an amount in the range of from 0.45 to 1.75% by, based on the total weight of the Alpinia galanga extract.

In case the anti-dandruff composition, as described above, comprises an Alpinia galanga extract which comprises said 1-acetoxychavicol acetate (I), the composition preferably comprises said extract in an amount the range of from 0.1 to 20% by weight, more preferably in an amount in the range of from 0.5 to 10% by weight, more preferably in an amount in the range of from 1 to 5% by weight, more preferably in an amount in the range of from 1.5 to 2.5%. by weight, such as in amount of about 2% by weight, based on the total weight of the anti-dandruff composition, wherein the Alpinia galanga extract preferably comprises 1-acetoxychavicol acetate (I) in an amount the range of from in an amount the range of from 0.1 to 10% by weight, more preferably in an amount in the range of from 0.1 to 5% by weight, more preferably in an amount in the range of from 0.3 to 3% by weight, more preferably in an amount in the range of from 0.45 to 1.75% by, based on the total weight of the Alpinia galanga extract.

Preferably, the extract is obtained from roots of Alpinia galanga, preferably of Zingiberaceae (common name: Thai Ginger, Great Galangal). Alpinia galanga, frequently also referred to as Languas galangal, belongs to the ginger family (Zingiberaceae). Usually, it is cultivated and grows wild in Asia. The herb is rhizomatic, about in height with oblong glabrous leaves and greenish white flowers. The fruits are orange-red capsules. In the context of the present invention, the term “Alpinia galanga” refers to any variety of Alpinia galanga. Preferably, the variety may be grown anywhere in the world.

Step (a)

Extraction, Maceration or Percolation

According to one preferred embodiment of the invention, the method comprises a step (a) as described above, wherein Alpinia galanga is contacted with a liquid S1.

The contacting in step (a) is preferably carried out by extraction, maceration or percolation, more preferably the Alpinia galanga is extracted with the liquid S1 or the contacting in (a) is carried out by a maceration process. The choice of the respective method usually depends on the nature of liquid S1 which is employed in step (a).

Maceration:

According to one preferred embodiment of the invention, the contacting in step (a) is carried out by maceration. Thus, the present invention also relates to a method, as described above, and a Alpinia galanga extract comprising 1-acetoxychavicol acetate (I) obtained or obtainable by said method, as described above, wherein the contacting in (a) is carried out by a maceration.

In this case, the liquid S1 preferably comprises a plant oil. The term “plant oil” refers to lipids obtained from plant sources.

Preferably the plant oil is an edible oil, more preferably a colorless edible oil.

Preferably, the plant oil is selected from the group consisting of safflower oil, sesame oil, sunflower oil, soy bean oil, olive oil rapeseed oil and mixtures thereof, more preferably the plant oil is selected from the group consisting of safflower oil, sunflower oil, rapeseed oil and mixtures thereof.

More preferably S1 consists of a plant oil, preferably a edible plant oil, more preferably wherein said oil is colorless, most preferably wherein said oil is selected from the group consisting of safflower oil, sesame oil, sunflower oil, soy bean oil, olive oil rapeseed oil and mixtures thereof, more preferably from the group consisting of safflower oil, sunflower oil, rapeseed oil and mixtures thereof.

In case the contacting is carried out by maceration, S1 and the Alpinia galanga material is preferably allowed to stand for a time in the range of from 30 min to 7 days, preferably in the range of from 24 h to 6 days, more preferably about 2 to 4 days, in particular at a temperature in the range of from 10 to 70° C., more preferably at a temperature in the range of from 40 to 60° C., most preferably around 50° C.

In case step (a) is carried out by maceration using a plant oil as solvent S1, the method preferably does not comprise steps (c) and (d), more preferably the method consist of steps (a) and (b).

Extraction:

According to a further preferred embodiment, the contacting in step (a) is carried out by extraction. Thus, the present invention also relates to a method, as described above, and an Alpinia galanga extract obtained or obtainable by said method, as described above, wherein in (a) Alpinia galanga is extracted with the liquid S1.

There are no particular restriction as to the extraction procedure, thus, any extraction method known to those skilled in the art, such as ultrasonic assisted extraction, soxhlet extraction, microwave assisted extraction and the like, may be used. Preferably the extraction is carried out at a temperature in the range of from 0° C. to 100° C., preferably in the range of from 10° C. to 40° C., more preferably at room temperature.

In this case, the liquid S1 preferably comprises an organic solvent, more preferably an organic solvent selected from the group consisting of heptane, iso-propanol, ethanol, methanol, acetone, water and mixtures thereof, more preferably of the group consisting of heptane, iso-propanol and mixtures thereof.

Preferably S1 comprises heptane in an amount of at least 60% by weight, more preferably of at least 80% by weight, more preferably of at least 99.9% by weight, in based on the total weight of S1.

Preferably S1 comprises less than 0.1% by weight of further components in total, preferably less than 0.05% by weight, based on the total weight of the solvent S1.

Thus, the present invention also relates to a method as described above and a composition obtained or obtainable by said method, as described above, wherein in (a) Alpinia galanga is extracted with the liquid S1, S1 comprises heptane in an amount of at at least 60% by weight, more preferably of at least 80% by weight, more preferably of at least 99.9% by weight, based on the total weight of S1.

Preferably, the ratio of amount of Alpinia galanga (weight) to solvent S1 (weight) is in the range of from 1:2 to 1 : 20, more preferably in the range of about 1:5.

The extraction can be carried out in one or more extraction steps. Preferably a multi-stage extraction is carried out in which a multiplicity of separating stages connected in series is used.

As described above, in step (a) a liquid phase L1 is formed, wherein said liquid phase comprises 1-acetoxychavicol acetate (I) and S1. Further a solid residue R0 is obtained, said solid residue being the remaining solid material of Alpinia galanga.

Step (b)

After the extraction, described above, the liquid phase L1 is separated from the solid residue R0.

The separation step may be carried out by any suitable method known to those skilled in the art. According to one embodiment of the invention, the separation is carried out by filtration. The term “filtration” or “filtering” refers to the process of removing essentially all, preferably all, of the solid residue R0, which may be present as suspended particles, from the liquid phase by passing the composition through one or more membranes or filters.

According to one preferred embodiment the obtained liquid phase L1, or an optionally concentrated and/or further purified L1 correspond to the Alpinia galanga extract. In this case S1 preferably comprises, in particular consists of a plant oil. According to this embodiment, the method for preparing Alpinia galanga preferably comprises the steps
(a) providing Alpinia galanga, more preferably a root of Alpinia Galanga, and contacting the Alpinia galanga with a liquid S1 thereby forming a liquid phase L1 comprising 1-acetoxychavicol acetate (I), and a solid residue R0;
(b) separating L1 from R0;
(g) optionally concentrating and/or purifying L1
   to give the Alpinia galanga extract.

Thus, in this case, the method preferably does not comprise steps (c) and (d), more preferably the method consist of steps (a) and (b).

Thus, in one aspect, the present invention also relates to a method for the preparation of an Alpinia galanga extract and an Alpinia galanga extract obtainable or obtained by said method, said method comprising
(a) providing Alpinia galanga, more preferably a root of Alpinia galanga, and contacting the Alpinia galanga with a liquid S1 thereby forming a liquid phase L1 and a solid residue R0; the liquid S1 being a plant oil,
(b) separating L1 from R0;
(g) optionally concentrating and/or purifying L1
   to give the Alpinia galanga extract.

Thus, according to this embodiment, L1, optionally the concentrated and/or purified L1, corresponds to the Alpinia galanga extract.

As described above, in step (g), L1 may be subjected to one or more further purification or work-up steps such as concentration of the extract and/or purifying the extract, e.g. filtering the extract to remove any undissolved material, to finally give the Alpinia galanga extract.

According an alternative embodiment of the invention, the method comprises the steps (and does not comprise step (g)):
(c) drying L1 to give a residue R1;
(d) dissolving the R1 in a liquid S2.

In this case S1 preferably comprises an organic solvent. Thus, in another aspect, the present invention also relates to a method for the preparation of an Alpinia galanga extract and an Alpinia galanga extract obtainable or obtained by said method, said method comprising
(a) providing Alpinia galanga, more preferably a root of Alpinia galanga, and contacting the Alpinia galanga with a liquid S1 thereby forming a liquid phase L1 and a solid residue R0; the liquid S1 being an organic solvent,
(b) separating L1 from R0;
(c) drying L1 to give a residue R1;
(d) dissolving the R1 in a liquid S2
   to give the Alpinia galanga extract

Optional Step c)

According to a further embodiment of the invention, L1 may, optional after further purification steps, be concentrated, in particular evaporated to dryness as mentioned above, thus e.g. by air drying, spray drying, vacuum oven drying, fluid-bed drying or freeze-drying to give residue R1. In the case in which L1 is evaporated to dryness, the residue R1 corresponds to the dry Alpinia galanga extract mentioned above.

Subsequent to the evaporating step, the method may comprise further steps, such as, e.g. at least one purification step and/or at least one homogenization step.

R1 thus comprises 1-acetoxychavicol acetate (I) in dry form.

Optional Step d)

As described above, according to one embodiment of the invention, residue R1, is preferably re-dissolved in a liquid S2, with S2 preferably comprising an organic solvent or a plant oil, more, preferably a plant oil, more preferably a plant oil selected from the group consisting of safflower oil, sunflower oil, olive oil, rapeseed oil and mixtures thereof, more preferably from the group consisting of safflower oil, sunflower oil, rapeseed oil and mixtures thereof.

It was surprisingly found that 1-acetoxychavicol acetate (I) is particularly stable in the obtained extract a plant oil is used as S1 and/or S2. The term “stable” in this context means that at least 90%, in particular essentially all of 1-acetoxychavicol acetate (I) present within the composition is stable and degraded or oxidized. It has been surprisingly found that the decrease of the peak area of 1-acetoxychavicol (HPLC UV 220 nm) in safflower oil with 3% lipophilic extract and in maceration oil with stabiliser (ascorbic acid palmitate or Butylhydroxyanisol) was lower than 10% over twelve (12) weeks under storage at 40° C. (see examples).

Without being bound to any theory, it is assumed that the presence of unsaturated fatty acids in S2 may avoid or diminish any oxidative stress which often occurs when using usual methods known in the art and which may adversely affect the active ingredients in the Alpinia galanga.

Thus, the present invention also relates to a method for preparing a Alpinia galanga extract comprising
(a) providing Alpinia galanga, more preferably a branch and/or a root of Alpinia galanga, and contacting the Alpinia galanga with a liquid S1 thereby forming a liquid phase L1 and a solid residue R0;
(b) separating L1 from R0;
(c) drying L1 to give a residue R1,
(d) dissolving the R1 in a liquid S2,
   to give the Alpinia galanga extract.

Preferably, the ratio of amount of R1 (weight) to liquid S2 (weight) is in the range of from 1:2 to 1:20, more preferably in the range of about 1:5.

Subsequent to the dissolving step d), the method may comprise further steps, such as, e.g. at least one purification step, such as filtering.

The anti-dandruff composition, described above, or the anti-dandruff composition obtainable by the method as described above, preferably comprises S1 or S2 in an amount in the range of from 0.1 to 10% by weight, more preferably in an amount in the range of from 0.5 to 5% by weigh and even more preferably in an amount in the range of from 1 to 3% by weight, based on the total weight of the composition.

Co-Pressing

According to a further preferred embodiment, the present invention relates to a method and to an anti-dandruff composition obtained or obtainable by said method, said method comprising
(a1) providing Alpinia galanga, and contacting Alpinia galanga with oil seeds;
(a2) subjecting the mixture according to (i) to co-pressing to give a liquid phase L1 and a solid residue R0;
(a3) separating L1 from R0
   to give the Alpinia galanga extract.

It has been surprisingly found that a particularly stable anti-dandruff composition comprising a high concentration 1-acetoxychavicol acetate (I), and a low concentration of fatty acids derived from Alpinia galanga is obtained when using the above mentioned method comprising the steps (a1) to (a3).

Without being bound to any theory, it is assumed that the presence of unsaturated fatty acids in L1 may avoid or diminish any oxidative stress which often occurs when using usual methods known in the art and which may adversely affect the active ingredients in the Alpinia galanga.

Further, the amount of 1-acetoxychavicol acetate (I) is substantially higher in the vegetable oils produced by the process described in this embodiment than can be achieved by a maceration process.

Preferably, the amount of 1-acetoxychavicol acetate (I) is in the vegetable oils produced by the process described in this embodiment is at least 0.45% by weight, preferably in the range of from 0.45 to 1.75% by weight, more preferably in the range of from 1.0 to 1.75% by weight, based on the total weight of the Alpinia galanga extract.

As described above, in step (a2) a liquid phase L1 is formed, wherein said liquid phase comprises 1-acetoxychavicol acetate (I) derived from Alpinia galanga and a plant oil derived from the respective oils seeds. It has to be understood that L1 may comprise various further substances derived from Alpinia galanga, however, preferably at least 99% by weight of all substances derived from Alpinia galanga, in particular the only substance derived from Alpinia galanga are/is acetoxychavicol acetate.

Further a solid residue R0 is obtained, said solid residue being the remaining solid material of Alpinia galanga and of the employed oil seeds.

According to a preferred embodiment, the oils seeds are selected from the group consisting of kernel seed, rape seed, sesame seed, sunflower seed and mixtures thereof. In particular, sunflower seeds are used.

Thus, the present invention also relates to a method and to an anti-dandruff composition obtained or obtainable by said method, said method comprising
(a1)) providing Alpinia galanga, and contacting Alpinia galanga with sunflower seeds;
(a2) subjecting the mixture according to (i) to co-pressing to give a liquid phase L1 and a solid residue R0;
(a3) separating L1 from R0
   to give the Alpinia galanga extract.

In step (a1), the Alpinia galanga material is preferably mixed with the oil seeds. The weight ratio of Alpinia galanga material to oils seed is preferably in the range of from 70:1 to 1:70.

The Alpinia galanga material is preferably provided in pieces having a maximum diameter of about 30 nm, such as in the range of from 1 nm to 20 nm. The provision of the Alpinia galanga material in step (a1) thus may comprise a step of chopping or shredding the Alpinia galanga material into pieces having a size in the range mentioned above.

It is to be understood that in this step, further components may be added such as further plant material, e.g. chopped or shredded parts of fragrant or aromatic plants such as, for and others may be used for the production of scented or fragrant enriched vegetable oils.

Furthermore, as additional component, further plant material comprising e.g. antifungal active ingredients to be extracted from this material when using the above mentioned method, may be used in this step.

As mentioned above, the oil seed and the Alpinia galanga material and optionally the additional components are co pressed in step (a2), preferably at a suitable temperature with a suitable pressure. Preferably only Alpinia galanga and the oilseeds are co-pressed in step (a2). A suitable co-pressing procedure is e.g. described in US 2002/0028272.

Preferably, the Alpinia galanga material is only compressed to a limited degree. The level of pressure exerted is such that while it does lead to breaking open or rupturing the plant cells it does not destroy them.

The temperature during step (a2) is kept at a level which ensures that no thermal damage of the components of the composition, in particular of 1-acetoxychavicol acetate (I) occurs. Preferably the temperature is in the range of from 10 to 70° C., more preferably in the range of from 15 to 50° C., even more preferably in the range of from 20 to 30° C. It is to be understood that during this step, the temperature may be varied or held essentially constant. Preferably the temperature is kept essentially constant.

Step (a2) is preferably carried out in an oil press, for instance, such that its enthalpy, i.e. temperature as a result of the exerted pressure, does not exceed 60° C. The flow conditions generated in the cylinder of the oil press cause the oil extracted from the oilseeds to be repressed at the nozzle aperture of the oil press thus washing the oil soluble ingredients from the plant material. Advantageously, the compression cylinder of the oil press is encased by a container for receiving the oil and protecting it from detrimental ambient effects such as the oxygen in the air and especially for preventing escape of the highly volatile ingredients or essential oils.

Further Components of the Anti-Dandruff Composition:

The anti-dandruff composition may be provided in various forms, such as in form of a solution (e.g. aqueous, aqueous-alcoholic or alcoholic solution), an emulsion of the water-in-oil (W/O) or oil-in-water (O/W) type or a multiple emulsion, for example of the water-in-oil-in-water (W/O/W) or oil-in-water-in-oil (O/W/O) type (in each case also in the form of silicone emulsions), a hydrodispersion or lipodispersion or a nanoemulsion. Further advantageous forms of administration of the composition according to the invention are creams, ointments, hydrodispersions, lotions, tinctures, pump sprays, aerosol sprays, aqueous solutions, cleansing substrates and the like.

The pharmaceutical or cosmetic composition can in principle be prepared by combining all ingredients at suitable conditions known to those skilled in the art using any method known to those skilled in the art. In principle, any suitable order of adding the ingredients may be used. Every mixture obtained during the preparation process may be e.g. stirred and/or homogenized. In case a homogenization is carried out, this homogenization is carried out with a thorax mixer.

Preferably, the anti-dandruff composition, described above, is a nanoemulsion.

The term “nanoemulsion,” as used herein, includes dispersions or droplets, as well as other lipid structures that can form as a result of hydrophobic forces that drive apolar residues (i.e., long hydrocarbon chains) away from water and drive polar head groups toward water, when a water immiscible oily phase is mixed with an aqueous phase. These other lipid structures include, but are not limited to, unilamellar, paucilamellar, and multilamellar lipid vesicles, micelles, and lamellar phases.

Preferably, the 1-acetoxychavicol (I) or the Alpinia galanga extract is present in the lipid phase of the nanoemulsion, in particular dissolved in the liquid phase L1, respectively. Most preferably, the lipid phase is present in form of droplets.

Preferably, the anti-dandruff composition described above is a nanoemulsion comprising
(i) an oily phase comprising L1 and at least 90%, preferably at least 95%, more preferably essentially all, of the total amount of 1-acetoxychavicol contained in the anti-dandruff composition,
(ii) an aqueous phase.

Diluent

Preferably, besides L1, the composition comprises at least one further liquid, that is at least one diluent. The at least one diluent preferably comprises at least water. Further additional suitable diluents are, for example alcohols, diols or polyols, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products.

Preferably, the anti-dandruff composition comprises at least water as diluent. Preferably, the anti-dandruff composition comprises additionally glycerol.

Besides 1-acetoxychavicol acetate (I), the anti-dandruff composition preferably comprises at least one cosmetically and/or pharmaceutically acceptable carrier and/or excipient.

The choice of the suitable cosmetically and/or pharmaceutically acceptable carriers and/or excipients depends on the intended use of the composition. In general, the compositions of the invention may be formulated and provided in any suitable form which is advantageous and effective for consumer use.

The term “carrier or excipient” as used herein, means any suitable vehicle, which can be used to apply the present compositions preferably to the skin in a safe and effective manner. A suitable carrier is stable, i.e. e.g., incapable of reacting with other ingredients in the composition. The excipient and/or carrier must be “cosmetically and/or pharmaceutically acceptable” and “safe and effective” in the sense of being compatible with the other ingredients of the composition and not deleterious to the recipient thereof.

It is to be understood that the preparation of the pharmaceutical or cosmetic composition of the invention preferably takes place under GMP standardized conditions in order to ensure quality, pharmaceutical security, and effectiveness of the pharmaceutical or cosmetic composition. Further criteria for an ingredient being pharmaceutically or cosmetically acceptable can be derived from approval regulations by a regulatory agency or other generally recognized pharmacopoeias.

According to an embodiment of the present invention, the pharmaceutical or cosmetic composition according to the invention is prepared by mixing 1-acetoxychavicol avetate (I) or the Alpnia galanga extract described above with one or more cosmetically and/or pharmaceutically acceptable carriers and/or excipients.

The pharmaceutical or cosmetic composition can in principle be prepared by combining all ingredients at suitable conditions known to those skilled in the art using any method known to those skilled in the art. In principle, any suitable order of adding the ingredients may be used. Every mixture obtained during the preparation process may be e.g. stirred and/or homogenized. In case a homogenization is carried out, this homogenization is carried out with a thorax mixer.

By way of example, the following cosmetically and/or pharmaceutically acceptable carrier and/or excipient may be mentioned: Antioxidants, perfume oils, foaming agents, surfactants, agents for preventing foaming, coloring agents, fragrances, thickening agents, chelating agents, such as sodium phytate, emulsifiers, humectants, oils, waxes or other conventional constituents of a cosmetic formulation, such as preferably alcohols, polyols, polymers, foam stabilizers, electrolytes, organic solvents or silicone derivatives, further anti-dandruff agents, anti-inflammatory agents, caring agents, stabilizers and the like.

Stabilizers and Antioxidants:

Optionally the pharmaceutical or cosmetic composition comprises at least one antioxidant and/or stabilizer, such as ascorbic acid palmitate, butylhydroxy toluene, butylhydroxy anisole, citric acid, potassium sorbate, biofavoic acid, glutathione, selenium, licopene, vitamin A, vitamin E, and vitamin C, as well as pyrrol derivatives, free radical scavengers obtainable from extracts of various plants, enzymes having antioxidant properties such as superoxide dismutases and glutathione peroxidases, and the like.

According to a preferred embodiment, the anti-dandruff composition, as described above, comprises at least one stabilizer, preferably selected from the group consisting of ascorbic acid palmitate, butylhydroxy anisol, citric acid, potassium sorbate and mixtures thereof.

Anti-Inflammatory Agents

Because dandruff formation is mostly accompanied by inflammatory processes, which manifest themselves especially in pronounced redness and itching of the scalp, the anti-dandruff composition of the invention optionally also contains at least one anti-inflammatory and/or redness- and/or itching-alleviating active ingredients. Any anti-inflammatory or redness- and/or itching-alleviating active ingredients suitable or conventional for cosmetic and/or dermatological applications can be used. By way of example, the following agents may be mentioned: oxicams, preferably piroxicam ortenoxicam; salicylates, preferably aspirin, disalcid, solprin or fendosal; acetic acid derivatives, preferably diclofenac, fenclofenac, indomethacin, sulindac, tolmetin or clindanac; fenamates, preferably mefenamic, meclofenamic, flufenamic or niflumic; propionic acid derivatives, preferably ibuprofen, naproxen, benoxaprofen, or pyrazoles, such as phenylbutazone, oxyphenylbutazone, febrazone or azapropazone, as well as plant extracts, such as extracts from camomile, aloe vera, Commiphora species, Rubia species, willow, willow-herb, oats, Calendula, arnica, St. John's wort, honeysuckle, rosemary, Melissa, ginger, Passiflora incamata, witch hazel, Pueraria, Dianthus or Echinacea, as well as pure substances such as, inter alia, bisabolol, apigenin, apigenin-7-glucoside, rosemary acid, boswellic acid, phytosterols, glycyrrhizinic acid, glabridin, licochalcone A, gingerols and anthranilic acid amides. Further ingredients of the corticosteroid type, preferably hydrocortisone, hydrocortisone derivatives such as hydrocortisone 17-butyrate, dexamethasone, dexamethasone phosphate, methylprednisolone or cortisone, may be mentioned.

Humectants

Further, the anti-dandruff compositions according to the invention may preferably comprise at least one humectant, more preferably an humectant selected from the group consisting of: sodium lactate; urea; urea derivatives; alcohols, preferably glycerol, further diols such as propylene glycol, hexylene glycol, 1,2-pentanediol or 1,2-hexanediol; collagen; elastin or hyaluronic acid; diacyl adipate; petrolatum; urocanic acid; lecithin; panthenol; phytantriol; lycopene; (pseudo) ceramides; glycosphingolipids; cholesterol; phytosterols; chitosan; chondroitin sulfate; lanolin; lanolin esters; amino acids; alpha-hydroxy acids (e.g. citric acid, lactic acid, malic acid) and derivatives thereof; mono-, di- and oligo-saccharides, preferably glucose, galactose, fructose, mannose, fruit sugars and lactose; polysugars, alpha-hydroxy fatty acids; triterpenic acids, preferably betulic acid or ursolic acid or algae extracts.

Caring Substances

Further, the anti-dandruff composition may comprise caring substances such as in particular N-acylsphingosines (fatty acid amides of sphingosine, so called ceramides) or synthetic analogues of such lipids (so-called pseudoceramides), which markedly improve the water-retaining capacity of the stratum corneum, phospholipids, for example soya lecithin, vaseline, waxes, paraffin and silicone oils; the latter include inter alia dialkyl- and alkylaryl-siloxanes such as dimethylpolysiloxane and methylphenylpoly-siloxane, as well as alkoxylated and quaternised derivatives thereof.

Further Anti-Dandruff Agents

According to a preferred embodiment of the invention, the anti-dandruff composition comprises, in addition to 1-acetoxychavicol acetate at least one further anti-dandruff agent. According to an alternative proffered embodiment, the anti-dandruff composition comprises, in addition to 1-acetoxychavicol acetate no further anti-dandruff agent.

Suitable, non-limiting examples of anti-dandruff particulates include: pyridinethione salts, azoles, selenium sulfide, particulate sulfur, and mixtures thereof. Preferred are pyridinethione salts, in particular zinc pyrithione. Such anti-dandruff agents should be physically and chemically compatible with the essential components of the composition, and should not otherwise unduly impair product stability, aesthetics or performance.

Pyridinethione anti-dandruff agents are described, for example, in U.S. Pat. Nos. 2,809,971; 3,236,733; 3,753,196; 3,761,418; 4,345,080; 4,323,683; 4,379,753; and 4,470,982. Suitable selenium sulfide compounds are described, for example, in U.S. Pat. Nos. 2,694,668; 3,152,046; 4,089,945; and 4,885,107

In case, the anti dandruff composition comprises at least one further anti-dandruff agent or pharmaceutically or cosmetically acceptable salt thereof, this anti dandruff-agent is preferably selected from the group consisting of climbazole. benzimidazole, benzothiazole, bifonazole, butaconazole nitrate, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, thioconazole, diazoles, triazoles, terconazole, itraconazole, 1-hydroxy-2-pyrithione salts, zinc pyrithione, coal tar, sulfur, selenium sulfides, aluminum chloride, octopirox (INCI: Piroctone Olamine), cyclopiroxolamines, undecylenic acid and its metal salts, potassium permanganate, sodium thiosulfate, propylene glycol, branched and unbranched aliphatic diols, branched and unbranched aliphatic polyols, urea, griseofulvin, 8-hydroxyquinoline, ciloquinol, thiobendazole, thiocarbamates, triclosan, haloprogin, polyenes, hydroxypyridone, morpholine, benzylamine, allylamines (e.g. terbinafine), tea tree oil, clove oil, coriander oil, palmarosa oil, thyme oil and cinnamon oil, ethereal oil of bitter orange, cinnamaldehyde, citronellic acid, farnesol, berberine, hinokitiol, tropolone, birch tar oils, ichthyol (sulfonated shale oil), Sensiva SC-50 (ethylhexyl glycerol), polyglycerol-3 caprylate, phenylethyl alcohol, 3-phenyl-l-propanol, vetikol (4-methyl-4-phenyl-2-pentanol), muguet alcohol (2,2-dimethyl-3-phenylpropanol), Elestab HP-100, azelaic acid, lyticase, octylisothiazalinone and iodopropynyl butyl carbamate (IPBC).

Surfactants:

According to a further preferred embodiment, the anti-dandruff composition according to the invention comprises at least one surfactant such as anionic, cationic, non-ionic, amphoteric surfactants and mixtures thereof. Suitable surfactants for use in cosmetical and pharmaceutical compositions are well known in the art. Surfactants are amphiphilic substances which are able to dissolve organic, non-polar substances in water. The hydrophilic components of a surfactant molecule are mostly polar functional groups, while the hydrophobic portions are generally non-polar hydrocarbon residues. Surfactants are generally classified according to the type and charge of the hydrophilic molecule portion. A distinction can be made between four groups, which have already been mentioned above, that is anionic surfactants, cationic surfactants, amphoteric surfactants and non-ionic surfactants. Anionic surfactants usually contain carboxylate, sulfate or sulfonate groups as functional groups. Cationic surfactants are almost exclusively characterized by the presence of a quaternary ammonium group. In aqueous solution they form positively charged organic ions in an acidic or neutral medium. Amphoteric surfactants contain both anionic and cationic groups and accordingly behave in aqueous solution like anionic or cationic surfactants, depending on the pH value. Typical examples for non-ionic surfactants are polyether chains.

Fragrances:

The pharmaceutical or cosmetic anti dandruff composition according to the invention may also further comprise at least one fragrance and/or at least one coloring agent. Fragrances and/or coloring agents well known to those skilled in the art may be used in effective amounts to impart the desired fragrance and color to the compositions of the invention.

Thus, the present invention also relates to an anti-dandruff composition, as described above, comprising at least one fragrance.

Any suitable perfumes may be used with no particular restriction which may be either synthetic perfumes or natural essential oils. The fragrance, for examples, include hydrocarbons, alcohols, phenols, aldehydes, and/or acetals, ketones and/or ketals, ethers, synthetic musks, acids, lactones, esters, halogen-containing compounds, and natural perfumes.

Specific examples hydrocarbons perfumes such as limonene, pinen, y-terepinen, and caryophyllene; alcohol perfumes such as phenyl ethyl alcohol, terepineol, bacdanol, geraniol, nerol, linarol, and cis-3-hexenol; aldehyde perfumes such as lilial, citral, aldehyde C-8, aldehyde C-9, aldehyde C-II, hexyl cynnamic aldehyde, vanillin, and heliotropin; keton perfumes such as yonon, rosephenone, woody flow, damasnin, isoe super; other perfume such as musks, eugenol and coumarin, in which compounds containing no sulfur or nitrogen atom are especially preferable; essential oils such as lemon oil, orange oil, and peppermint oil; and essences such as apple essence and strawberry essence. It is to be understood that, in case a fragrance is used, one or more fragrances may be used.

Conditioning Agents

The pharmaceutical or cosmetic anti dandruff composition according to the invention may also further comprise at least one conditioning agent. An conditioning agent is a material which is used to give a particular conditioning benefit to hair and/or skin. As example, silicones or organic conditioning oils (e.g. hydrocarbon oils, polyolefins, and fatty esters) or combinations thereof, may be mentioned.

Suitable organic conditioning oils for use as conditioning agents include, but are not limited to paraffin oil, mineral oil, saturated and unsaturated dodecane, saturated and unsaturated tridecane, saturated and unsaturated tetradecane, saturated and unsaturated pentadecane, saturated and unsaturated hexadecane, polybutene, polydecene, and mixtures thereof. Branched-chain isomers of these compounds, as well as of higher chain length hydrocarbons, can also be used, examples of which include highly branched, saturated or unsaturated, alkanes such as the permethyl-substituted isomers, e.g., the permethyl-substituted isomers of hexadecane and eicosane, such as 2,2,4,4,6,6,8,8dimethyl-10-methylundecane and 2,2,4,4,6,6-dimethyl-8methylnonane, available from Permethyl Corporation. Hydrocarbon polymers such as polybutene and polydecene. A preferred hydrocarbon polymer is polybutene, such as the ethoxylated glyceryl monostearate, 1,3-butylene glycol monostearate, 1,3-butylene glycol. Also suitable for use in the compositions herein are the conditioning agents described by the Procter & Gamble Company in U.S. Pat. Nos. 5,674,478, and 5,750,122, both of which are incorporated herein in their entirety by reference. Also suitable for use herein are those conditioning agents described in U.S. Pat. No. 4,529,586, U.S. Pat. No. 4,507,280, U.S. Pat. No. 4,663,158, U.S. Pat. No. 4,197,865, U.S. Pat. No. 4,217,914, U.S. Pat. No. 4,381,919, and U.S. Pat. No. 4,422,853.

Thickening Agents

The pharmaceutical or cosmetic anti dandruff composition according to the invention may also further comprise at least one suitable thickener. Suitable thickeners are the swelling agents customarily used for gel formation in galenic pharmacy.

Examples of suitable thickeners include natural organic thickeners, such as agar-agar, gelatin, gum arabic, a pectin, and the like, modified organic natural compounds, such as carboxymethylcellulose or cellulose ethers, or fully synthetic organic thickeners, such as poly arylic compounds, vinyl polymers, or poly ethers.

In some embodiments, the excipient can increase the smoothness or other properties of the composition. Such additives include, but are not limited to glycerin, propylene glycol, butylene glycol, esters, diacyl glycerol esters, and starch.

Furthermore, the thickeners may be selected from algin; carbomers cellulose gum; cetearyl alcohol, cocamide DEA, dextrin; gelatin; hydroxyethylcellulose; hydroxypropylcellulose; hydroxypropyl methylcellulose; magnesium aluminum silicate; myristyl alcohol; oat flour; oleamide DEA; oleyl alcohol; PEG-7M; PEG-14M; PEG-9OM; stearamide DEA; stearamide MEA; stearyl alcohol; tragacanth gum; wheat starch; xanthan gum; wherein DEA is diethanolamine, and MEA is monoethanolamine. Alternatively or in addition thereto, thickeners used the composition of the present invention may comprise one or more of aluminum stearates; beeswax; candelilla wax; carnauba; ceresin; cetearyl alcohol; cetyl alcohol; cholesterol; hydrated silica; hydrogenated castor oil; hydrogenated cottonseed oil; hydrogenated soybean oil; hydrogenated tallow glyceride; hydrogenated vegetable oil; hydroxypropyl cellulose; lanolin alcohol; myristyl alcohol; octytdodecyl stearoyl sulfate; oeyl alcohol; ozokerite; microcystalline wax; paraffin, pentaerythrityl tetraoctanoate; polyacrylamide; polybutene; polyethylene; propylene glycol dicaprylate; propylene glycol dipelargonate; stearalkonium hectorite; stearyl alcohol; stearyl stearate; synthetic beeswax; trihydroxystearin; trilinolein; tristearin; zinc stearate; and the like.

According to a preferred embodiment of the invention, the composition according to the invention comprises at least one thickener, such as glycerol or xanthane gum.

Chelating Agents

The pharmaceutical or cosmetic anti dandruff composition according to the invention may also further comprise at least one chelating agents, i.e. an agent that may bind metal ions or metallic compounds. Preferred chelating agents include tetrasodium- and trisodium-ethylenediaminetetraacetate, sodium phytate and the like.

Optional Further Components

Further vitamins and amino acids such as: water soluble vitamins such as vitamin B1, B2, B6, B12, C, pantothenic acid, pantothenyl ethyl ether, panthenol, biotin, and their derivatives, water soluble amino acids such as asparagine, alanin, indole, glutamic acid and their salts, water insoluble vitamins such as vitamin A, D, E, and their derivatives, water insoluble amino acids such as tyrosine, tryptamine, and their salts, should be mentioned.

According to a further aspect, the composition may also comprise protein hydrolysates, such as an almond partial hydrolysate.

Use/Method of Treatment

As described above, the present invention also relates to an anti-dandruff composition, as described above, for use in treating or preventing Malassezia induced dandruff formation and/or Malassezia induced itching of the skin, and to the use of an anti-dandruff composition, as described above for treating or preventing Malassezia induced dandruff formation and/or Malassezia induced itching of the skin. Preferably, the present invention also relates to an anti-dandruff composition, as described above, for use in treating or preventing Malassezia induced dandruff formation, and to the use of an anti-dandruff composition, as described above for treating or preventing Malassezia induced dandruff formation. More preferably, the present invention also relates to an anti-dandruff composition, as described above, for use in treating or preventing Malassezia induced dandruff formation and Malassezia induced itching of the skin, and to the use of an anti-dandruff composition, as described above for treating or preventing Malassezia induced dandruff formation and Malassezia induced itching of the skin.

Further, the present invention also relates to an anti-dandruff composition, as described above, for use in treating or preventing Malassezia induced itching of the skin, and to the use of an anti-dandruff composition, as described above for treating or preventing Malassezia induced itching of the skin.

According to a further aspect, the present invention also relates to a method of treating Malassezia induced dandruff formation and/or itching of the skin comprising applying an anti-dandruff composition, as described above.

The method of the invention is in particular suitable for the treatment of individuals suffering from dandruff formation on the scalp. The anti-dandruff composition is preferably applied topically, more preferably in form of a shampoo.

The method according to the invention may comprise a single administration. According to an alternative embodiment, the administration is repeated several times, such as 2 to 3 times daily, for several days, such as e.g. up to 6 weeks. Unexpectedly, it was found that 1-acetoxychavicol acetate (I) or an Alpinia galanga extract comprising the same is capable of reducing dandruff formation on the scalp while being non-toxic and essentially non-irritating when applied to the skin. Thus, after using the treatment of the invention, the presence of dandruff is diminished or even no dandruff is existent anymore. Further, the itching is diminished.

In the following, by way of example, the following especially preferred embodiments of the invention are mentioned:
1. An anti-dandruff composition, preferably for use in treating or preventing Malassezia induced dandruff formation or Malassezia induced itching of the skin or Malassezia induced dandruff formation and Malassezia induced itching of the skin, the composition comprising 1-acetoxychavicol acetate according to formula (I)

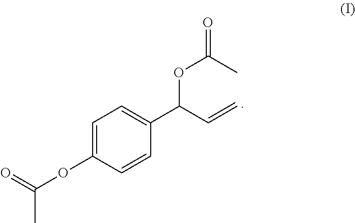 [SMILES: C=CC(OC(C)=O)c1ccc(OC(C)=O)cc1]

2. The anti-dandruff composition according to embodiment 1, comprising an Alpinia galanga extract which comprises said 1-acetoxychavicol acetate (I).
3. The anti-dandruff composition according to embodiment 2 which comprises the Alpinia galanga extract in an amount the range of from 0.1 to 20% by weight, based on the total weight of the anti-dandruff composition.
4. The anti-dandruff composition according to any one of embodiments 1 to 3, wherein the Alpinia galanga extract is obtainable or obtained by a process comprising
   (a) providing Alpinia galanga, and contacting the Alpinia galanga with a liquid S1 thereby forming a liquid phase L1 and a solid residue R0;
   (b) separating L1 from R0;
   (c) optionally drying L1 to give a solid residue R1;
   (d) optionally dissolving R1 in a liquid S2
to give the Alpinia galanga extract.
5. The anti-dandruff composition according to embodiment 4, wherein in (a), Alpinia galanga is extracted with S1.
6. The composition according to embodiment 4 or 5, wherein S1 is an organic solvent, preferably selected from the group consisting of heptane, iso-propanol, ethanol, methanol, acetone, water and mixtures thereof and mixtures thereof, more preferably heptane or iso-propanol or a mixture thereof.
7. The anti-dandruff composition according to embodiment 4 or 5, wherein S1 is a plant oil, preferably a plant oil selected from the group consisting of safflower oil, sunflower oil, olive oil, rapeseed oil and mixtures thereof.
8. The anti-dandruff composition according to embodiment 6 or 7, wherein the extracting in (a) comprises a maceration.
9. The anti-dandruff composition according to any one of embodiments 1 to 3, wherein the Alpinia galanga extract is obtainable or obtained by a process comprising
   (a1) providing Alpinia galanga, and contacting Alpinia galanga with oil seeds;
   (a2) subjecting the mixture according to (i) to co-pressing to give a liquid phase L1 and a solid residue R0;
   (a3) separating L1 from R0
to give the Alpinia galanga extract.
10. The anti-dandruff composition according to embodiment 9, wherein the oil seeds are sunflower seeds.
11. The anti-dandruff composition according to any one of embodiments 1 to 10 further comprising at least one stabilizer, preferably selected from the group consisting of ascorbic acid palmitate, butylhydroxy anisol, citric acid, potassium sorbate and mixtures thereof.
12. The anti-dandruff composition according to any one of embodiments 1 to 11, wherein the composition is a nanoemulsion comprising
   (i) an oily phase comprising L1 and at least 90%, preferably at least 95%, more preferably essentially all, of the total amount of 1-acetoxychavicol contained in the anti-dandruff composition,
   (ii) an aqueous phase.
13. The anti-dandruff composition according any one of embodiments 1 to 12, further comprising at least one further anti-dandruff agent or pharmaceutically or cosmetically acceptable salt thereof, preferably selected from the group consisting of climbazole, benzimidazole, benzothiazole, bifonazole, butaconazole nitrate, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, thioconazole, diazoles, triazoles, terconazole, itraconazole, 1-hydroxy-2-pyrithione salts, zinc pyrithione, coal tar, sulfur, selenium sulfides, aluminum chloride, octopirox (INCI: Piroctone Olamine), cyclopiroxolamines, undecylenic acid and its metal salts, potassium permanganate, sodium thiosulfate, propylene glycol, branched and unbranched aliphatic diols, branched and unbranched aliphatic polyols, urea, griseofulvin, 8-hydroxyquinoline, ciloquinol, thiobendazole, thiocarbamates, triclosan, haloprogin, polyenes, hydroxypyridone, morpholine, benzylamine, allylamines (e.g. terbinafine), tea tree oil, clove oil, coriander oil, palmarosa oil, thyme oil and cinnamon oil, ethereal oil of bitter orange, cinnamaldehyde, citronellic acid, farnesol, berberine, hinokitiol, tropolone, birch tar oils, ichthyol (sulfonated shale oil), Sensiva SC-50 (ethylhexyl glycerol), polyglycerol-3 caprylate, phenylethyl alcohol, 3-phenyl-l-propanol, vetikol (4-methyl-4-phenyl-2-pentanol), muguet alcohol (2,2-dimethyl-3-phenylpropanol), Elestab HP-100, azelaic acid, lyticase, octylisothiazalinone and iodopropynyl butyl carbamate (IPBC).
14. The anti-dandruff composition according any one of embodiments 1 to 13, further comprising one or more cosmetically and/or pharmaceutically acceptable carriers and/or excipients.
15. The anti-dandruff composition according any one of embodiments 1 to 14, further comprising at least one fragrance.
16. A method for the preparation of an Alpinia galanga extract, comprising
   (a) providing Alpinia galanga, more preferably a root of Alpinia galanga, and contacting the Alpinia galanga with a liquid S1 thereby forming a liquid phase L1 and a solid residue R0;
   (b) separating L1 from R0;
   (c) optionally drying L1 to give a residue R1;
   (d) optionally dissolving the R1 in a liquid S2 to give the Alpinia galanga extract.
17. The method according to embodiment 16, wherein in (a) Alpinia galanga is extracted with S1.
18. The method according to embodiment 16 or 17, wherein S1 is an organic solvent, preferably selected from the group consisting heptane, iso-propanol, ethanol, methanol, acetone, water and mixtures thereof and mixtures thereof, more preferably heptane or iso-propanol or a mixture thereof.
19. The method according to embodiment 16 or 17, wherein S1 is a plant oil, preferably a plant oil selected from the group consisting of safflower oil, sunflower oil, olive oil, rapeseed oil and mixtures thereof.
20. The method according to embodiment 19, wherein the extracting in (a) comprises a maceration.
21. A method for the preparation of an Alpinia galanga extract, comprising
   (a1) providing Alpinia galanga, more preferably the a root of Alpinia galanga, and contacting the Alpinia galanga with oil seeds;
   (a2) subjecting the mixture according to (a1) to co-pressing to give a liquid phase L1 and a solid residue R0,
   (a3) separating L1 from R0
to give the Alpinia galanga extract.
22. The method according to embodiment 21, wherein the oil seeds are sunflower seeds.
23. An Alpinia galanga extract obtainable or obtained by the method according to any one of embodiments 16 to 22, preferably for use in treating or preventing Malassezia induced dandruff formation and Malassezia induced itching of the skin.
24. An anti-dandruff composition according any one of embodiments 1 to 14 for use in treating or preventing Malassezia, in particular Malassezia furfur, induced dandruff formation.
25. An anti-dandruff composition according any one of embodiments 1 to 14 for use in treating or preventing Malassezia, in particular Malassezia furfur, induced itching of the skin.
26. An anti-dandruff composition according any one of embodiments 1 to 14 for use in treating or preventing Malassezia, in particular Malassezia furfur, induced dandruff formation and itching of the skin.
27. Use of an anti-dandruff composition according any one of embodiments 1 to 14 for treating or preventing Malassezia, in particular Malassezia furfur, induced dandruff formation.
28. Use of an anti-dandruff composition according any one of embodiments 1 to 14 for treating or preventing Malassezia, in particular Malassezia furfur, induced itching of the skin.
29. Use of an anti-dandruff composition according any one of embodiments 1 to 14 for treating or preventing Malassezia, in particular Malassezia furfur, induced dandruff formation and itching of the skin.
30. Method of treating Malassezia induced dandruff formation comprising applying an anti-dandruff composition according any one of embodiments 1 to 14.
31. Method of treating Malassezia induced itching of the skin comprising applying an anti-dandruff composition according any one of embodiments 1 to 14.
32. Method of treating Malassezia induced dandruff formation comprising applying an anti-dandruff composition according any one of embodiments 1 to 14.

### FIGURES

FIG. 1: Analysis of the prepared according to example 1 (n-heptane)

FIG. 2: NMR spectrum of fraction (medium polar part of the extract), see example 4

FIG. 3: Schematic drawing of the mid-log assay and corresponding results.

Cells are cultivated to half-maximal OD580 before test compounds are added (arrow). Value of OD580 at time of sample application is set 100%. A) ineffective test compound show no inhibition of growth. Level-% is identical to control-level-%; B) test compound inhibits cell growth, level-%<Ctrl-level, >100%; C) stagnation; no further growth upon addition of test compound, level-%=100%; D) cell-lysis; OD580 diminishes upon addition of test compound; level-%<100%. Blue diamonds, untreated control cells; pink rectangles, cells treated with test compounds.

FIG. 4: NMR spectrum of 1-acetoxychavicol

### EXAMPLES

### Examples

The examples which follow are intended to illustrate the present invention without limiting it. Unless indicated otherwise, all amounts, parts and percentages are based on the weight and the total amount, or on the total weight and the total amount, or on the total weight of the preparations.

Alpinia galanga was obtained from Alfred Galke GmbH, Gittelde/Harz; No 815204: Galangae Rad. kbA pulv.

### Example 1

### Generation of Lipophilic Extract

The dried and finely grounded Alpinia galanga material was extracted twice with fivefold n-heptane in total (sonicated for about 15 minutes, shaked for about 30 minutes and afterwards centrifugated), yielding approx. 2% extract regarding the quantity of plant material.

### Example 2

### Generation of Maceration Oil Using Safflower Oil

The dried and finely grounded Alpinia galanga material was extracted with fourfold safflower oil for three days at 50° C. with stirring occasionally and afterwards centrifugated, yielding approx. 60% maceration oil regarding the added quantity of safflower oil.

### Example 3

### Generation of Oil Containing Dandruff Active Principle by Co-Pressing Procedure

The dried and coarsely grounded Alpinia galanga material (6 to 8 mm particle size) in addition of threefold quantity of peeled sunflower seed was processed by the SPE (Short-Press-Extraction)-technology, yielding approx. 50 to 60% received Alpinia galanga oil regarding the added quantity of peeled sunflower seed.

### Example 4

### Profiling of Extract, Bioassay Guided Fractionation

The dried and finely grounded Alpinia galanga material was extracted (MTB (methyl-tert-buthylether)/methanol (1:1), 2×methanol) and the yielded extract fractionated by RP (reversed-phase) HPLC yielding three (3) fractions.

Sample preparation: 1 g extract were dissolved in 3 ml DMSO, 1 ml water were added and filtrated, liquid injection of the filtrate

Method description:

Column: SelectB 12 μm 50×25 mm with pre-column

Detection: HPLC-ELSD-UV (254 nm)

Mobile Phase: A: H2O; B: ACN:MeOH (1:1)

Gradient: 10% B 0.5 min; from 10% B to 100% B in 0.8 min, 6.3 min 100% B

Flow: 28 ml/min

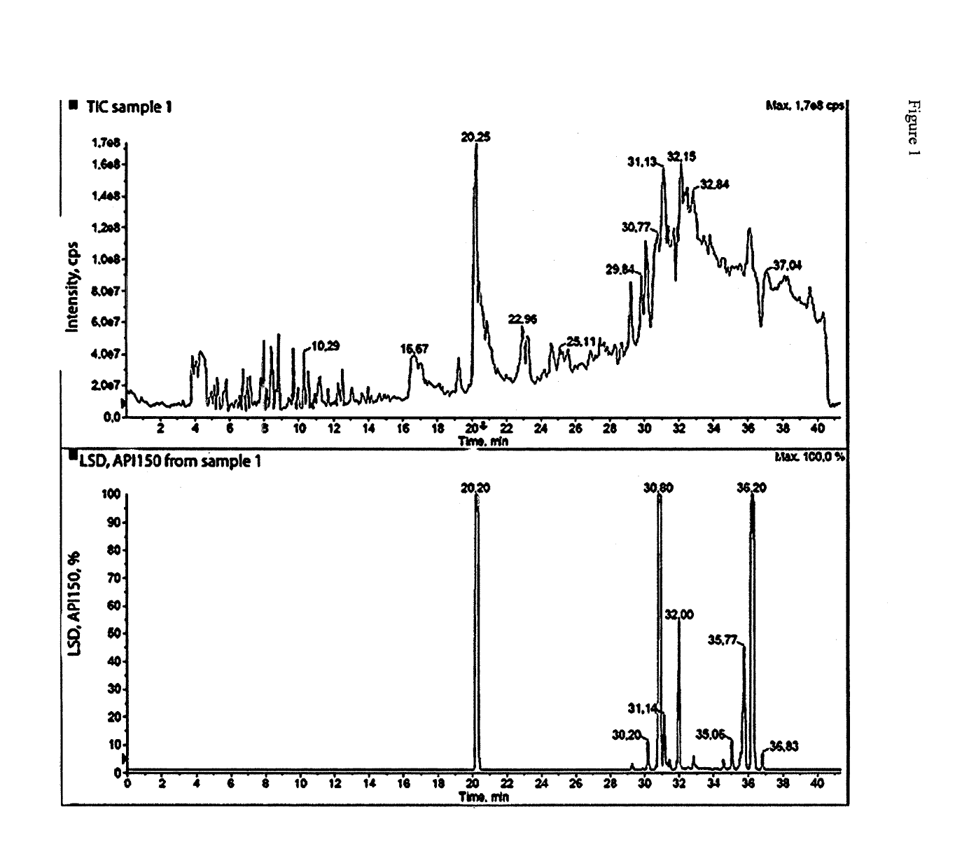

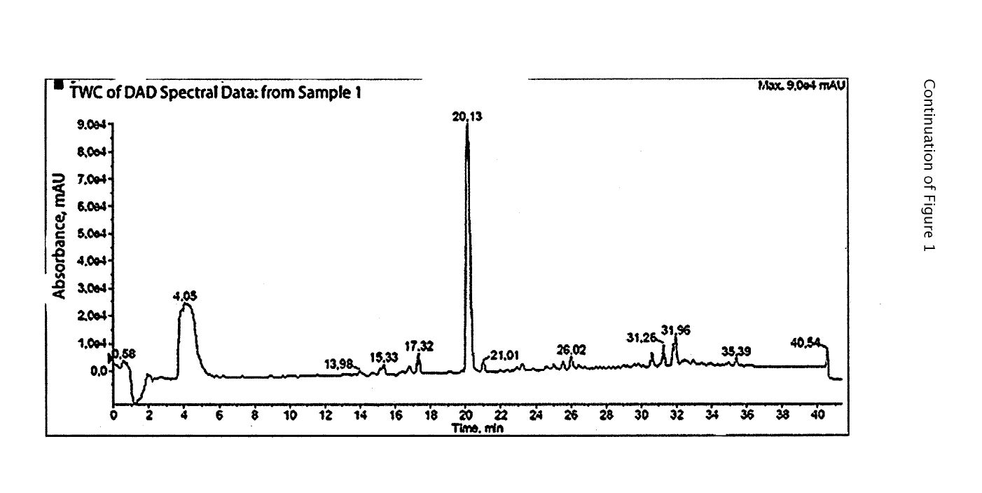

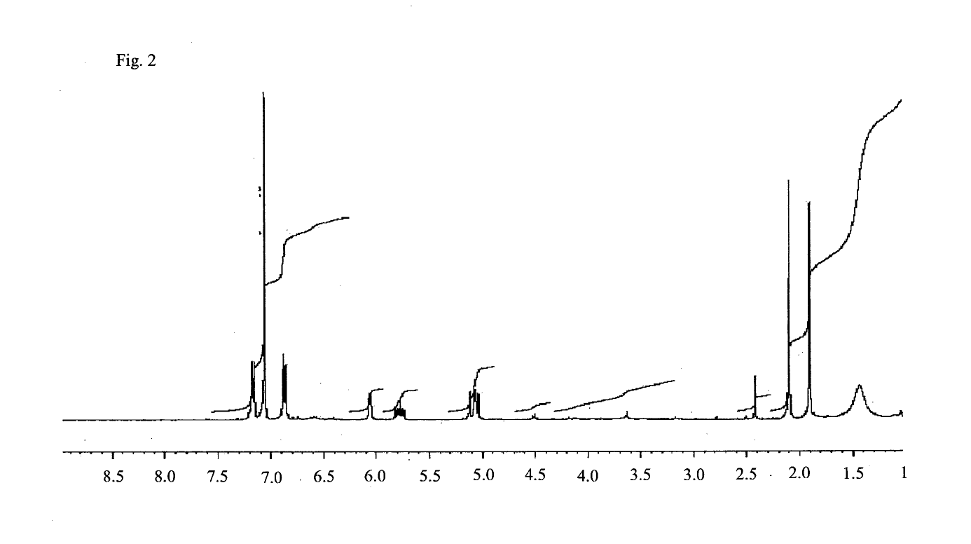

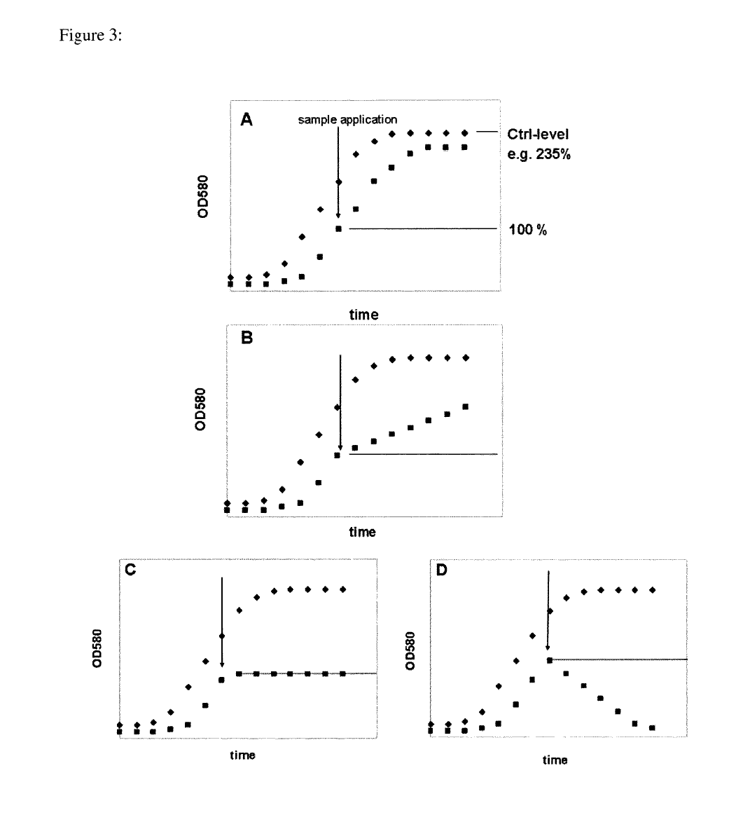

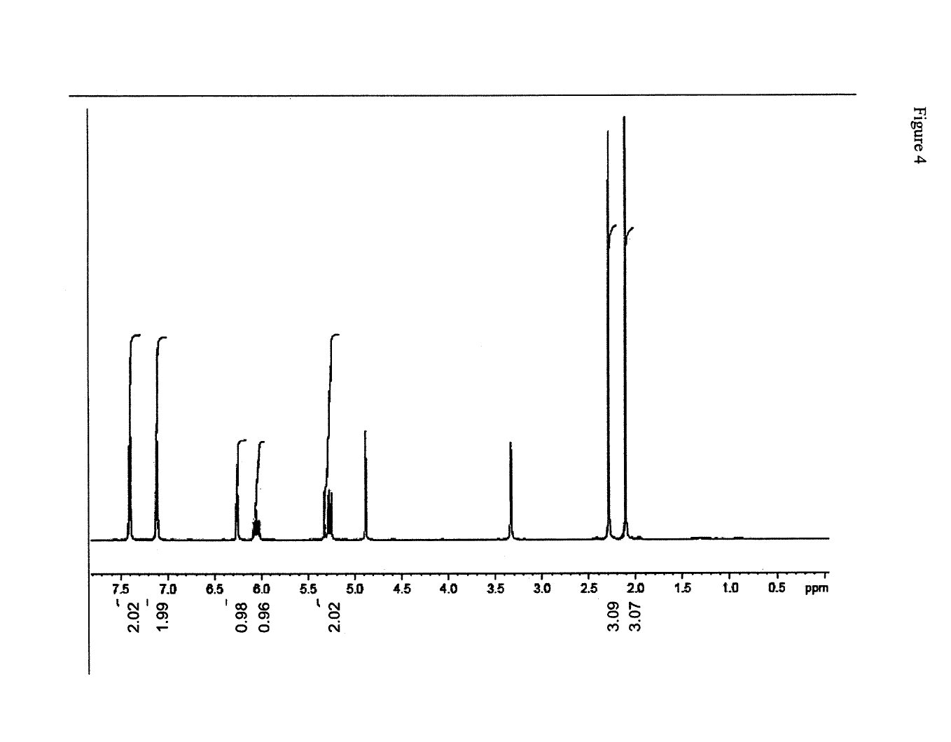

The results of the analysis of example 1 are shown in FIG. 1

### Example 5

### Stability Assessment of 1-Acetoxychavicol Acetate Using Different Stabilizers

### Example 5.1

### Preparation of Samples for Storage Stability Testing

(a) Safflower Oil with 3% Lipophilic Extract
1.5 g lipophilic extract was dissolved in 50 g safflower oil at 80° C. and split in 10-g-batches:
To the first batch 10 mg/ml ascorbic acid palmitate was added as a stabilizer and the sample was stored at 40° C.
Add to the second batch 10 mg/ml BHA (Butylhydroxyanisol) as a stabilizer and the sample was stored at 40° C.
Another batch was stored as a reference at −18° C.

(b) Maceration Oil
Add to 10 g maceration oil 100 mg ascorbic acid palmitate as a stabilizer and the sample was stored at 40° C.
Add to 10 g maceration oil 100 mg BHA (Butylhydroxyanisol) as a stabilizer and the sample was stored at 40° C.
10 g maceration oil were stored as a reference at −18° C.

(c) Preparation of Oil Samples for HPLC Analysis:

0.5 ml of the samples prepared according to example 5.1 (a) and (b) were extracted with 3 ml methanole/water (9:1), centrifugated and the supernatant was used for the analysis in example 5.2

### Example 5.2

### Analysis of the Samples Prepared According to Example 5.1(c)) Using HPLC Method

(a) Method Used
Column: Kromasil C18, 125×4 mm with pre-column
Detection: UV (220 nm)
Mobile Phase: A: water with 5 mM ammonium formiate and 0.1% formic acid;
B:acetonitrile/methanol=1:1, 5 mM ammonium formiate and 0.1% formic acid
Gradient: from 60% B to 80% B in 30 min
Flow: 0.8 ml/min
Retention time of the 1-acetoxychavicol peak is 14.2 (±0.2) min.

(b) Results
The decrease of the peak area of 1-acetoxychavicol (HPLC UV 220 nm) in safflower oil with 3% lipophilic extract and in maceration oil with stabiliser (ascorbic acid palmitate or Butylhydroxyanisol) was lower than 10% over twelve (12) weeks under storage at 40° C.

### Example 6

Studies on the microbial action of a lipophilic extract of Alpinia galanga (1-acetoxychavicol 50 weight-%) against Malassezia furfur (Synonym: Pityrosporum ovale)

Assay Principle

M.spp. are usually characterized by their morphology, guanosine-cytosine content, secretion products and their dependency on certain lipid sources. Under laboratory conditions (96 well plates) it takes 7 days until e.g. M. sympodialis reaches stationary growth phase. Within this period planktonic growth can be quantified by spectroscopic means and a heterogeneous biofilm can be detected but poorly be quantified by standard crystal violet incorporation. However, within these heterogeneous biofilms patches of residual cells form which are highly adhesive and therefore stick to the plates even after several washing steps.

For the screening of potential anti-microbial or anti-biofilm effective compounds a classical toxicity assay in liquid culture (planktonic growth) and a residual growth assay (biofilm) have been used.

Materials & Methods

Routine cultivation of M. sympodialis: M. sympodialis (former M. furfur) CBS 1878 was obtained from the Centraalbureau voor Schimmelcultures Fungal Biodiversity Centre. It was routinely grown on Dixon-agar plates in a humid atmosphere (“wet box”) at 33° C., or in Sabouraud-Maltose (SM) broth complemented with Tween80 and Glycerin-monostearate for liquid cultures in shake-flasks (300 ml operated with 50 ml medium) with agitation (150 rpm).

General Assay Conditions
1. Strain: Malassezia sympodialis ATCC 42132
2. Growth conditions:
Sabouraud-medium supplemented with dextrose (20 g/l), Tween 80 (1%) and glycerol-monostearat (emulsifier; 0.05%). Static incubation in flat-bottom 96-well microtiterplates (total volume 340 μl, working volume 200 μl; surface area 148 mm2). Incubation at 33° C. in humid atmosphere (wet-box) to avoid dessication. Incubation periods vary according to the assay format. Suspensions used for inoculation were adjusted to an optical density (OD580) of 0.1
3. Reference substances (“benchmarks”)
Results of test compounds are compared to climabzole-formulations (Crinipan AD, >98%) and preparations of Zinc Pyrithione (ZPT, >48.2%)
4. Controls
Sterile media was used as negative control; cells cultivated in absence of any test compound but in presence of equal amounts of DMSO compared to samples (see below) were regarded as positive control (unrestricted growth)
5. All compounds (test-samples and references) are dissolved in DMSO. DMSO was shown to have no negative effect on cell growth and is present in equal amounts in all samples, including untreated control samples.
6. Unless otherwise stated, test compound concentration was 12.5 μg/ml

“Classic assay” for Susceptibility Testing of Planktonically Growing Cells

Routine susceptibility testing of M. sympodialis in high throughput format was done by simultaneous addition of test compounds and inoculum. Seven days after inoculation and when planktonic growth of the untreated control ceased, the effect of the test compounds was evaluated by comparison of the turbidity (optical density, OD580) of treated and untreated samples (control; 100%).

The classic assay is the platform to determine dose-response relationships, where serial dilutions of compounds are evaluated. For description of these relationships, values for the minimal effective concentration and the concentration effective in reducing cell proliferation by 50% (IC50) are generated. The MEC is defined as the lowest concentration of test compound that has at least 90% effectiveness compared to the maximum effect observed

‘Mid-Log Assay’:

For the so called “mid-log assay”, cells are grown to half-maximum optical density before test compounds are added. The OD580 (t₀) is recorded and cells are incubated further until the untreated samples (control) do not show further increase in turbidity (stationary growth phase; t_{end}). Numbers for OD580 of untreated control samples and samples treated with test compounds are compared to t₀-value, which is defined as the 100% level. The value for OD580 of the untreated control sample in stationary phase (end of experiment) is defined as the “control-level” (for unrestricted growth). Values for control-levels are solely dependent on t₀-values and therefore vary with each experiment; thus, they cannot be standardised. Control level values are always higher than 100%, ideally show values of about 200%, but might be even higher. The effectiveness of test compounds is given as level-% and has to be compared to the respective control-levels. Level-% values for test compounds might be close to control-level values (no effect), vary between 100% and control-level (inhibition), are close to 100% (stagnation), or fall below 100% (cell-lysis).

Exponentially growing cells (mid-log) are in a defined active state with respect to proliferation and metabolic activity. Cell numbers are significantly higher compared to the “classic assay”, so that only highly active compounds will have an inhibitory effect on planktonic growth of M. sympodialis. Moreover, the mode of inhibition caused by the test compound can be determined (see above).

RGA—‘Residual Growth Assay’:

During growth in microtiter plates, M. sympodialis readily forms a heterogeneous biofilm including highly adhesive patches. Patches were also observed in samples of inhibited planktonic growth. In order to determine the effectiveness of test compounds to inhibit repopulation of the bulk phase by persister cells surviving in patches, the residual growth assay was developed.

M. sympodialis is grown in presence of test compounds for 7 days or until the untreated control enters stationary growth phase. Planktonic growth is measured (OD580), and planktonic cells, inconsistent biofilm cells and test compounds are removed by 3 subsequent washing steps, each applying fresh medium while resuspending the medium. After washing, only highly adhesive cells, mostly organised in patches, remain. Following on, the ability of those patches to cause repopulation of the sterile bulk phase is monitored by spectroscopy twice a day (8/16 h intervals) over a period of 4 days or until stationary phase of the positive control is reached. Optical densities of samples and controls are compared to determine the effectiveness of test compounds. Optical densities of untreated samples gave values for unrestricted growth. Effectiveness of compounds tested are given in % reduction based on the OD-value of the untreated sample which reflects 100%.

| | |
|---|---|
| planktonic growth | |
| Primary Screening | 96.8% inhibition |
| Seondary Screening | 92.7% inhibition |
| Minimimal Effective Concetration | 12.5 μg/ml |
| IC₅₀-Determination: | |
| Alpinia galanga extract | 7.4 μg/ml |
| Crinipan | 0.2 μg/ml |
| Zinc Pyrithione | 12.4 μg/ml |
| Mid-log Assay: | |
| Level-% | 63% → cell lysis |
| Control-level % | 125% |
| biofilm-growth mode | |
| Residual Growth Assay | 99.6% inhibition |

### Example 7

### Studies on the Microbial Action of 1-Acetoxychavicol Against Malassezia furfur (Synonym: Pityrosporum ovale)

Bioactivity of BAS-0203 (NAT0-399 304)

Materials, methods and assay principles see example 6

planktonic growth

| Classic Assay: | |
|---|---|
| 1-acetoxychavicol | 61% inhibition |
| Crinipan | 93% inhibition |
| Zinc Pyrithione | 73% inhibition |
| Mid-log Assay: | |
| Level-% | 379 |
| Control-level % | 399 |
| %-inhbition compared | |
| to benchmark: | |
| 1-acetoxychavicol | 11% |
| Crinipan | 66% |
| Zinc Pyrithione | 16% |

### Example 8

Example formulation for preparations for the prophylaxis and/or treatment of Malessezia-induced dandruff comprise a composition comprising 1-acetoxychavicol and/or a liphophilic extract of Alpinia galanga containing a dandruff-reducing amount of 1-acetoxychavicol as well as one, two, three, four, five, six, seven, eight, nine, ten, eleven, or twelve further anti-dandruff agents selected from the group consisting of climbazole, ketoconazole, zinc pyrithione, coal tar, sulphur, selenium disulfide, octopirox (INCI: piroctone olamine), cyclopiroxolamine, undeylenic acid and the metal salts thereof, ichthyol (sulonated shale oil), Sensiva SC-50 (ethylhexylglycerin) and/or vetikol (4-methyl-4-phenyl-2-pentanol).

### Example 9

### Body Care Shampoo for Treating or Preventing Dandruff

| Phase | No. | Ingredients | wt. % |
|---|---|---|---|
| A | 1 | Glycerol 86.5% PHEUR herbal, supplier: | 5.00 |
| | | Fauth&Co.KG | |
| | 2 | Keltrol (xanthane gum) CG-SFT, supplier: Rahn AG | 1.50 |
| | 3 | water, desalted; low germ content | 56.30 |
| | 4 | Dermofeel PA-3 (sodium phytate); supplier: Dr. | 0.10 |
| | | Straetmans | |
| | 5 | Potassium Sorbate Granulate E202, Supplier: | 0.25 |
| | | Azelis Kosmetik GmbH | |
| | 6 | Sodium Benzoat gran. Purox S, Supplier: Azelis | 0.25 |
| | | Kosmetik GmbH | |
| B | 7 | Euperlan Green (dispersion of waxes), Fa. Azelis | 3.00 |
| | | Kosmetik GmbH | |
| | 8 | Amisoft CS-22 (anionic surfactant; Sodium Cocoyl | 8.00 |
| | | Glutamate (and) Disodium Cocoyl Glutamate 25%), | |
| | | Fa. Rahn AG | |
| | 9 | Plantacare 818 UP (non-ionic surfactant); supplier: | 20.00 |
| | | Azelis Kosmetik GmbH | |
| | 10 | Lamesoft PO 65 (Coco-Glucoside (and) Glyceryl | 2.00 |
| | | Oleate), supplier: Azelis Kosmetik GmbH | |
| | 11 | Gluadin Almond Benz (almond partial hydrolysate), | 0.50 |
| | | Fa. Azelis Kosmetik GmbH | |
| | 12 | Cosmaderm T-70 NON GMO (hyaluron), Cosphatec | 0.10 |
| | | GmbH | |
| | 13 | Oil of Alpinia galanga (made by co-pressing with | 2.00 |
| | | sunflower seed) | |
| C | 14 | Citric acid 1xH2O, 50% in water, Fa. Merck KGaA | 1.00 |

Phase A:
Ingredients No. 1 + 2 were mixed at room temperature.
Ingredients No. 2-6 were added an homogenized in vacuo for 10 min
Phase B:
Ingredients No. 7-13 were mixed in a separate vessel, and were sub-
sequently added to phase A, the combined phases were homogenized in
vacuo for 10 min.
Phase C:
Ingredient No. 6-8 were added tot he homogenized Phase A/B and
were homogenized in vacuo for 10 min and cooled down to 30° C.
Phase B:
Ingredients No. 14 was used to adjust the pH 5.

### Example 10

### Study to Investigate the Efficacy of the Body Care Shampoo According to Example 9

To assess the efficacy of an anti-dandruff shampoo comprising 1-acetoxychavicol, a study with 20 volunteers suffering from dandruff was carried out. In this study, the effect of the shampoo on the occurrence of the dandruff as well as the cosmetic acceptance and safety was recorded.

In the study, twelve females and eight males were provided with the shampoo which was labeled “organic cosmetic for daily use”. The volunteers were at least 18 years of age, clinically healthy, had full head of hair and moderate to very strong dandruff (rated 3, 4 or 5 on a 6-point dandruff intensity scale as described below). These volunteers did not suffer from any other skin diseases, such as atopic dermatitis, or psoriasis or any other medical condition requiring systemic medical treatment or which may interfere with the study. Further, they had no planned medical treatment during the study, where non-pregnant and did not use any other anti-dandruff shampoos within three months prior to the study.

The volunteers were provided with the shampoo with the instruction to use the shampoo once daily, i.e. to apply a small amount (hazelnut size) of the shampoo to the wet hair and prewash the hair, then rinse and reapply to wash a second time. The second time, they were instructed to thoroughly massage the product into the hair and the scalp and leave the product on the scalp and in the hair for two minutes before rinsing thoroughly. The shampoo was tested once daily for a period of four weeks. Prior to the start of the study, the subjects completed a two-week wash-in phase, once daily application, using a standard shampoo provided to them without any actives against dandruff. All volunteers used the same shampoo.

At the beginning of the study, as well as after two weeks and four weeks, the subjects' scalp were assessed by two independent trained evaluators and the volunteers were further asked to create their subjective perceptions of dandruff intensity and itching. The last test product used was always scheduled to occur in a time period of 16-24 hours before reporting for evaluation. Except for limited use of hair styling products (gel, foam, hair spray), no other cosmetic products (shampoos, conditioners etc.) were allowed on the scalp/in the hair during the two weeks wash-in phase and during the whole study. Dyeing or coloring of the hair was prohibited. To allow for a proper assessment, styling products were not to be used on the days of assessing the scalp.

Methods

(a) Professional Evaluation

The dandruff intensity was assessed on days 0, 14 and 28 and rated according a 6-point dandruff intensity scale, with the following meaning: 0=no, 1=barely visible, 2=minimal, 3=moderate, 4=strong and 5=very strong.

(b) Interviews

The volunteers were asked to rate the product using the following scoring system on days 0, 14 and 28:

For dandruff formation: 0=no, 1=minimal, 2=moderate, 3=strong; and

For itching: 0=no, 1=minimal, 2=moderate, 3=strong.

Results and Discussion

(a) Professional Evaluation

Evaluated were the dandruff intensity scores as collected independently by the two evaluators on days 14 and 28 as compared to the initial scores collected after the two-week wash-in period.

TABLE 1
Improvement in dandruff intensity (mean scores)

| | Start | 2 weeks | 4 weeks |
|---|---|---|---|
| Evaluator 1 | 3.95 | 3.55 | 3.25 |
| Evaluator 2 | 3.95 | 3.45 | 3.3 |

Improvements in dandruff intensity were observed as follows:

TABLE 2
Improvement in dandruff intensity (% of subjects)

| | | 2 weeks | 4 weeks |
|---|---|---|---|
| Evaluator 1 | 1 category | 35% | 60% |
| | 2 categories | 5% | 10% |
| Evaluator 2 | 1 category | 40% | 55% |
| | 2 categories | 10% | 10% |

As may be taken from these results, the results from both professional evaluators clearly correlate with each other and show that the anti-dandruff shampoo shows indeed efficacy in reducing dandruff (correlation coefficient between 0.80 and 0.93). The decrease as found by both evaluators is at day 14 and day 28 significant as compared to the initial condition (p<0.05; Wilcoxon Rank Test).

(b) Self-Evaluation of Dandruff Intensity, Itching and Hair State

Evaluated were the dandruff and itching intensity scores according to the 4-point scales described above on days 14 and 28 as compared to the initial score collected after the two-week wash-in period.

The following results were achieved:

TABLE 3
Improvement in dandruff and itching intensity (mean scores)

| | Start | 2 weeks | 4 weeks |
|---|---|---|---|
| Dandruff | 2.65 | 2.4 | 2.05 |
| Itching | 1.15 | 0.85 | 0.7 |

Thus, the improvement of dandruff intensity and itching intensity were observed as follows:

TABLE 4
Improvement in dandruff intensity (% of subjects)

| | 2 weeks | 4 weeks |
|---|---|---|
| 1 category - dandruff | 25% | 50% |
| 1 category - itching | 60% | 70% |
| (adjusted percentage) | | |

The results on dandruff and itching clearly show that the anti-dandruff shampoo is efficient in reducing dandruff and itching at every time-point of scoring, i.e. at days 14 and 28. The decrease in both, i.e. dandruff and itching, is at both days statistically significant compared to the initial condition (p<0.05; Wilcoxon Rank Test).

Thus, the improvement in dandruff intensity correlates well with the professional evaluation scores.

As may be taken from these result, surprisingly, compositions comprising a low concentration of an Alpinia galanga extract (2 weight % based on the total weight of the body shampoo), thus a very low concentration of 1-acetoxychavicol (around 0.024 weight % based on the total weight of the body shampoo), already show a significant effect in reducing dandruff and further, advantageously, additionally in reducing itching of the scalp. Studies with higher concentrations of an Alpinia galanga extract and of 1-acetoxychavicol are envisaged.

1. An anti-dandruff composition comprising 1-acetoxychavicol acetate according to formula (I) for use in treating or preventing Malassezia induced dandruff formation and/or Malassezia induced itching of the skin

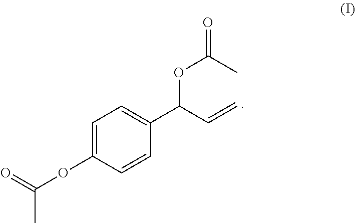 [SMILES: C=CC(OC(C)=O)c1ccc(OC(C)=O)cc1]

2. The anti-dandruff composition according to claim 1, comprising an Alpinia galanga extract which comprises said 1-acetoxychavicol acetate (I), preferably wherein the anti-dandruff composition comprises the Alpinia galanga extract in an amount the range of from 0.1 to 20% by weight, based on the total weight of the anti-dandruff composition.

3. The anti-dandruff composition according to claim 1, wherein the Alpinia galanga extract is obtainable or obtained by a process comprising:
(a) providing Alpinia Galanga, and contacting the Alpinia galanga with a liquid S1 thereby forming a liquid phase L1 and a solid residue R0;
(b) separating L1 from R0;
(c) optionally drying L1 to give a solid residue R1; and
(d) optionally dissolving R1 in a liquid S2
to give the Alpinia galanga extract.

4. The anti-dandruff composition according to claim 3, wherein in (a), Alpinia galanga is extracted with S1, preferably wherein S1 is:
an organic solvent, more preferably selected from the group consisting heptane, iso-propanol, ethanol, methanol, acetone, water and mixtures thereof and mixtures thereof, more preferably heptane or iso-propanol or a mixture thereof, or
a plant oil, preferably a plant oil selected from the group consisting of safflower oil, sunflower oil, olive oil, rapeseed oil and mixtures thereof,
preferably wherein the extracting in (a) comprises a maceration.

5. The anti-dandruff composition according to claim 1, wherein the Alpinia galanga extract is obtainable or obtained by a process comprising:
(a1) providing Alpinia Galanga, and contacting Alpinia galanga with oil seeds, preferably sunflower seeds;
(a2) subjecting the mixture according to (i) to co-pressing to give a liquid phase L1 and a solid residue R0;
(a3) separating L1 from R0
to give the Alpinia galanga extract.

6. The anti-dandruff composition according to claim 1, further comprising at least one stabilizer, preferably selected from the group consisting of ascorbic acid palmitate, butylhydroxy anisol, citric acid, potassium sorbate and mixtures thereof.

7. The anti-dandruff composition according to claim 1, wherein the composition is a nanoemulsion comprising:
(i) an oily phase comprising L1 and at least 90%, preferably at least 95%, more preferably essentially all, of the total amount of 1-acetoxychavicol comprised in the anti-dandruff composition, and
(ii) an aqeous phase.

8. The anti-dandruff composition according to claim 1, further comprising at least one further anti-dandruff agent or pharmaceutically or cosmetically acceptable salt thereof, preferably selected from the group consisting of climbazole. benzimidazole, benzothiazole, bifonazole, butaconazole nitrate, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, thioconazole, diazoles, triazoles, terconazole, itraconazole, 1-hydroxy-2-pyrithione salts, zinc pyrithione, coal tar, sulfur, selenium sulfides, aluminum chloride, octopirox (INCI: Piroctone Olamine), cyclopiroxolamines, undecylenic acid and its metal salts, potassium permanganate, sodium thiosulfate, propylene glycol, branched and unbranched aliphatic diols, branched and unbranched aliphatic polyols, urea, griseofulvin, 8-hydroxyquinoline, ciloquinol, thiobendazole, thiocarbamates, triclosan, haloprogin, polyenes, hydroxypyridone, morpholine, benzylamine, allylamines (e.g. terbinafine), tea tree oil, clove oil, coriander oil, palmarosa oil, thyme oil and cinnamon oil, ethereal oil of bitter orange, cinnamaldehyde, citronellic acid, farnesol, berberine, hinokitiol, tropolone, birch tar oils, ichthyol (sulfonated shale oil), Sensiva SC-50 (ethylhexyl glycerol), polyglycerol-3 caprylate, phenylethyl alcohol, 3-phenyl-l-propanol, vetikol (4-methyl-4-phenyl-2-pentanol), muguet alcohol (2,2-dimethyl-3-phenylpropanol), Elestab HP-100, azelaic acid, lyticase, octylisothiazalinone and iodopropynyl butyl carbamate (IPBC).

9. The anti-dandruff composition according to claim 1, further comprising one or more cosmetically and/or pharmaceutically acceptable carriers and/or excipients.

10. A method for the preparation of an Alpinia galanga extract, comprising:
(a) providing Alpinia Galanga, more preferably a root of Alpinia Galanga, and contacting the Alpinia galanga with a liquid S1 thereby forming a liquid phase L1 and a solid residue R0;
(b) separating L1 from R0;
(c) optionally drying L1 to give a residue R1;
(d) optionally dissolving the R1 in a liquid S2,
to give the Alpinia galanga extract.

11. The method according to claim 10, wherein in (a) Alpinia galanga is extracted with S1, preferably wherein S1 is:
an organic solvent, preferably selected from the group consisting heptane, iso-propanol, ethanol, methanol, acetone, water and mixtures thereof and mixtures thereof, more preferably heptane or iso-propanol or a mixture thereof, or
a plant oil, preferably a plant oil selected from the group consisting of safflower oil, sunflower oil, olive oil, rapeseed oil and mixtures thereof,
preferably wherein the extracting in (a) comprises a maceration.

12. A method for the preparation of an Alpinia galanga extract, comprising
(a1) providing Alpinia Galanga, more preferably the a root of Alpinia Galanga, and contacting the Alpinia galanga with oil seeds, preferably sunflower seeds;
(a2) subjecting the mixture according to (a1) to co-pressing to give a liquid phase L1 and a solid residue R0, and
(a3) separating L1 from R0,
to give the Alpinia galanga extract.

13. An Alpinia galanga extract obtainable or obtained by the method according to claim 10.

14. A method of treating or preventing Malassezia induced dandruff formation and/or Malassezia induced itching of the skin comprising applying to a subject in need an anti-dandruff composition according to claim 1.

15-16. (canceled)
